(19) United States
(12) Patent Application Publication
(10) Pub. No.: US 2011/0135724 A1
(43) Pub. Date: Jun. 9, 2011

(54) Ondansetron Orally Disintegrating Tablet Compositions for Prevention of Nausea and Vomiting

(75) Inventor: Gopi M. Venkatesh, Vandalia, OH (US)

(73) Assignee: Eurand, Inc., Vandalia, OH (US)

(21) Appl. No.: 12/956,249

(22) Filed: Nov. 30, 2010

Related U.S. Application Data
(60) Provisional application No. 61/265,233, filed on Nov. 30, 2009.

(51) Int. Cl. A61K 9/20; A61K 31/4178; A61P 1/08; B05D 1/00

(52) U.S. Cl. 424/465; 514/397; 427/2.21

(57) ABSTRACT

This invention is related to a pharmaceutical composition in the patient-friendly orally disintegrating tablet form comprising a weakly basic, selective serotonin 5-HT₃ blocking agent for the prevention of nausea and/or vomiting for up to 24 hrs postdosing in cancer patients prior to undergoing moderately emetogenic chemotherapy or partial or whole body radiotherapy or in subjects at moderate to high risk of postoperative or postdischarge nausea and/or vomiting prior to inpatient or outpatient ambulatory surgery. The unit dosage form comprising a multitude of immediate-release drug particles providing dissolution profiles similar to that of reference drug product, and one or more timed, pulsatile-release bead populations, comprising at least one organic acid, which solubilizes said weakly basic selective serotonin 5-HT₃ blocking agent prior to releasing it into the hostile intestinal environment, wherein the blocking agent is practically insoluble, is capable of delivering said antiemetic agent in patients in need thereof in a sustained-released fashion to be suitable for a once-daily dosing regimen.

### CROSS REFERENCE TO RELATED APPLICATIONS

The present application claims priority to U.S. Provisional Application No. 61/265,233, filed Nov. 30, 2009, the disclosure of which is herein incorporated by reference in its entirety for all purposes.

### BACKGROUND OF THE INVENTION

Nausea is an unpleasant feeling in the stomach that may or may not be followed by vomiting. Vomiting is the sudden, forceful expulsion of the stomach contents which may or may not be preceded by nausea. They very often occur together but can also occur independently of each other. It is most common in patients with cancer undergoing chemotherapy. Postoperative nausea and vomiting (PONV) and postdischarge nausea and vomiting (PDNV) are common post-surgical complications. PONV typically refers to nausea and vomiting which occurs after surgery, such as immediately after surgery. PDNV refers to post-surgical nausea and vomiting, but specifically refers to the nausea and vomiting occurring after the patient has been discharged, after the immediate effects of anesthesia have worn off and the patient is relatively ambulatory.

The chemical triggering zone (CTZ) for nausea and vomiting is located at the area postrema on the floor of the 4^{th} ventricle of the brain, and raised intracerebral pressure is thought to cause vomiting via increased pressure at the ventricle. The CTZ is extremely sensitive to emetic stimuli. Various neurotransmitter types and receptors have been implicated in nausea and vomiting, including serotonin, acetylcholine, dopamine, muscarine, neurokinin-1, histamine, opioid, and 5-HT₃. Stimulation of the vestibular-cochlear, glossopharyngeal, or vagus nerves may also be involved. Accordingly, the risk factors for nausea and vomiting are complex, and known antiemetic agents vary widely in their effectiveness.

Antiemetics are typically administered via an oral route prior to the start of moderately emetogenic chemotherapy or radiotherapy, or inpatient or outpatient ambulatory surgery, or intravenously during surgery (e.g., in the final stages of surgery) in order to have an immediate prophylactic effect, and are often not administered subsequently unless or until the patient experiences nausea and/or vomiting. In some cases, oral, immediate release antiemetics are administered. PONV and PDNV can result in patient discomfort (mild to severe), but can also have significant clinical consequences such as resulting in damage to delicate surgical sites, prolonging the time patients stay in post anesthesia care units, interrupting or delaying the administration of oral medications or fluid/food intake, and ultimately cause unplanned readmission or hospitalization following ambulatory surgery, thereby increasing medical costs (Kovac, A L. Drugs; 59(2): 213-243).

5-HT₃ receptor antagonists such as ondansetron are highly specific and selective for nausea and vomiting, and are known to be most effective when given orally prior to surgery, intravenously (IV) at the end of surgery, or IV after surgery in the early part (i.e., 0-2 hr period) of PONV. The recommended IV dose of ondansetron is 4 to 8 mg IV in adults, and 50 to 100 μg/kg in children. As a practical matter, it is difficult or inconvenient to administer IV antiemetics post-discharge. Oral administration is more convenient, less costly, and safer.

Ondansetron is currently available only as an immediate release tablet (e.g., conventional tablets, ZOFRAN Tablets, 4 and 8 mg (containing ondansetron HCl dihydrate equivalent to 4 and 8 mg of ondansetron, respectively), orally disintegrating tablets, ZOFRAN ODTs, 4 and 8 mg (containing ondansetron base), or an oral suspension, ZOFRAN Oral Solution (each 5 mL containing 5 mg of ondansetron HCl dihydrate equivalent to 4 mg of ondansetron). For immediate release dosage forms, the relatively short in-vivo half-life of ondansetron results in an ondansetron plasma concentration characterized by sharp peaks and troughs, thereby requiring that the dosage form be administered periodically in order to be effective over a 24-hour period.

For example, for prevention of nausea and vomiting associated with initial and repeat courses of moderately emetogenic cancer chemotherapy, the recommended oral dosage of ZOFRAN in pediatric patients at least 12 years of age and adults is one 8-mg ZOFRAN Tablet or one 8-mg ZOFRAN ODT Tablet or 10 mL (2 teaspoonfuls equivalent to 8 mg of ondansetron) of ZOFRAN Oral Solution given twice a day. The first dose should be administered 30 minutes before the start of emetogenic chemotherapy, with a subsequent dose 8 hours after the first dose. Oral administration twice a day (every 12 hours) should be continued for 1 to 2 days after completion of chemotherapy. For pediatric patients 4 through 11 years of age, the recommended dose is 4 mg of ondansetron given 3 times a day.

For prevention of nausea and/or vomiting associated with radiotherapy in patients receiving either total body irradiation, single high-dose fraction to the abdomen, or daily fractions to the abdomen, the recommended dose is 8-mg ondansetron administered 1 to 2 hours before radiotherapy and continued for 1 to 2 days after completion of radiotherapy.

For prevention of postoperative nausea and/or vomiting, the recommended dose is 16 mg of ondansetron administered 1 hour before induction of anesthesia. As with other antiemetics, routine prophylaxis is not recommended for patients in whom there is little expectation that nausea and/or vomiting will occur postoperatively. In patients where nausea and/or vomiting must be avoided postoperatively, ZOFRAN tablet, ODT or Oral Solution is recommended even where the incidence of postoperative nausea and/or vomiting is low.

However, the type of pharmacokinetic profile achieved in the dosing regimens recommended above is often associated with alternating periods of increased side effects and inefficacy as the plasma concentrations of drug cycle outside of the ideal therapeutic range. This cycling of drug plasma levels can result in the break through symptoms, i.e. nausea and vomiting. This makes the therapeutic effect unpredictable both between patients and upon repeated dosing. Repeat dosing schedules also pose other problems for patients who are distressed, experiencing nausea and vomiting, and may have difficulty swallowing. To these factors are added the noncompliance with administration schedules associated with repeat dosage schedules. All of these factors reduce the effectiveness of prophylactic oral doses of antiemetics. Accordingly, it would be advantageous to provide orally administrable once-daily dosage forms containing 5-HT₃ receptor antagonists such as ondansetron effective to prevent nausea and/or vomiting in cancer patients undergoing chemo- or radiotherapy or in subjects at moderate to high risk to PONV or PDNV in at least the first 24 hr period following surgery.

Difficulty in swallowing conventional tablets and capsules due to fear of choking, dysphagia, or actual pain, especially in cancer patients, is common among all age groups. For example, it is observed in about 35% of the general population, as well as an additional 30-40% of elderly institutionalized patients and 18-22% of all persons in long-term care facilities, many of whom are required to consume medications on a regular basis to maintain their quality of life. There are potential advantages of avoiding taking the medication with water by patients undergoing surgery (Gan, T J. et al. Anesth. Analg. 2002; 94: 1199-1200). Zofran ODT, a freeze-dried oral formulation based on the Zydis® technology, has shown superiority in terms of reduced incidence of nausea over placebo in patients after ambulatory surgery, outpatient gynecological laperoscopy (Table 1). All patients received a prophylactic dose of ondansetron 4 mg IV at induction. Prior to discharge, patients were randomly allocated to receive ondansetron ODT 8 mg or placebo tablet and a second dose 12 hrs after. Table 1 compares incidence of nausea, emesis, patient satisfaction, and acceptability of study drug. Patients rated the taste of the ondansetron ODT less favorably than the placebo ODT due to the bitter aftertaste of the drug.

TABLE 1
Incidence of pre-and post discharge Nausea and Vomiting

| | Ondansetron ODT | Placebo ODT |
|---|---|---|
| | (n = 30) | (n = 30) |
| Age (yrs) | 41 ± 14 | 37 ± 13 |
| Weight (kg) | 76 ± 11 | 73 ± 14 |
| Intraoperative fentanyl (μg) | 128 ± 52 | 137 ± 71 |
| Duration of anesthesia (min) | 83 ± 36 | 71 ± 34 |
| History of PONV/Motion sickness | 8 | 10 |
| (n) | | |
| Predischarge nausea (%) | 40 | 37 |
| Predischarge emesis (%) | 3 | 0 |
| PD rescue antiemetic use (%) | 33 | 30 |
| Post-discharge nausea (%) | 30 | 50 |
| Post-discharge nausea (%) | 3 | 23 |
| Severity of nausea (VRS*) | 0 (0-0) | 2 (0-10) |
| Acceptability of study drug (VRS) | 5.5 (1-10) | 10 (9-10) |

*→ verbal rating score (scale: 0-10)

The compositions of the present invention fill a currently unmet need for a once-daily, user-friendly antiemetic dosage form (e.g., an orally disintegrating tablet) that can be conveniently orally administered without grittiness and aftertaste issues, which will provide an immediate prophylactic effect as well as a continuing beneficial effect up to 24 hrs post-dosing.

### SUMMARY OF THE INVENTION

The present invention is directed to an orally disintegrating tablet (ODT) comprising a multiparticulate, selective serotonin 5-HT₃ blocking agent-containing pharmaceutical composition and rapidly dispersing microgranules. The rapidly dispersing microgranules comprise at least one super disintegrant and at least one sugar alcohol or a saccharide with a mean primary particle size of not more than 30 μm, and the multiparticulate, selective serotonin 5-HT₃ blocking agent-containing pharmaceutical composition comprises immediate-release beads with rapid release characteristics similar to that of the reference product, combined with one or more timed pulsatile-release (TPR) bead populations. The TPR beads comprise a TPR (lag-time) coating, a weakly basic selective serotonin 5-HT₃ blocking agent, an organic acid, and a sustained release membrane. Upon oral administration without water, the ODT of the present invention rapidly disintegrates in the oral cavity into a smooth (non-gritty), easy-to-swallow suspension, that can be easily swallowed by patients, e.g., patients at moderate to high risk of PONV/PDNV prior to undergoing inpatient or outpatient ambulatory surgery or by cancer patients prior to undergoing moderately emetogenic cancer chemotherapy, radiotherapy receiving total body irradiation, single high-dose fraction to the abdomen, or daily fractions to the abdomen for the prevention of nausea and/or vomiting for up to 24 hours post-dosing.

The method of making a once-daily dosage form as an orally disintegrating tablet of a selective serotonin 5-HT₃ blocking agent such as ondansetron HCl dehydrate is disclosed in one or more of the embodiments of the present invention, wherein Ondansetron ODT CR in accordance with the disclosures in U.S. 2007/0196491 and U.S. 2009/0232885, comprises rapid-release (RR)/immediate-release (IR) beads with rapid release characteristics similar to that of the reference product, Zofran® and one or more timed pulsatile-release (TPR) bead populations, wherein the TPR beads comprise a TPR (lag-time) coating, an organic acid layer with a sustained release membrane that not only prevents mixing between the organic acid in the inner layer and the drug in the outer layer, but also controls the rate of acid diffusion into the drug layer to synchronize the drug release with that of the acid. The rapid release drug particles and immediate release (IR) beads to be developed into TPR beads in accordance with the above invention comprising a selective serotonin 5-HT₃ blocking agent are designed in the form of beads comprising the drug (e.g., ondansetron HCl) layered on small particle size inert cores (e.g., smaller than 425 μm or more preferably less than 250 μm), or pellets comprising at least one pharmaceutically acceptable excipient and optionally an organic acid, granulated/extruded/spheronized or formed by controlled spheronization or powder layering using Granurex from Vector/Freund Corporation or the like, exhibiting rapid release similar to that of the reference product under discriminating dissolution conditions, i.e., USP Apparatus 2 in 500 mL buffer at pH 6.8.

In certain other embodiments of the present invention, the extended release ODT dosage form of the present invention comprises at least one TPR bead population with each bead comprising a sustained-release (SR) or a TPR coating disposed over an organic acid crystal, a polymeric binder layer comprising the selective serotonin 5-HT₃ antagonist, an optional sustained-release (SR) coating layer disposed over the drug layer, and/or an external TPR coating layer, in order to insure solubilization of the selective serotonin 5-HT₃ antagonist inside the coated bead prior to its release into the alkaline pH environment of the intestinal tract where the drug is practically insoluble, thereby providing a method of treating or preventing nausea and vomiting comprising orally administering to patients in need thereof a once-daily ODT dosage form prior to or following postoperative surgery, chemotherapy, or radiation therapy.

In one embodiment, the extended release dosage form of the present invention comprises timed, sustained-release (TSR) beads and IR beads; wherein each TSR bead comprises a core coated first with an SR layer and second with a TPR layer; the core comprises a selective serotonin 5-HT₃ antagonist and a pharmaceutically acceptable organic acid, wherein the selective serotonin 5-HT₃ antagonist and the pharmaceutically acceptable organic acid are separated from each other by an SR or TPR layer; the TPR layer comprises a water insoluble polymer and an enteric polymer; the SR layer comprises a water insoluble polymer; and the IR beads, each bead comprising the selective serotonin 5-HT₃ antagonist, releases at least about 80 wt. % of the selective serotonin 5-HT₃ antagonist in about 15 minutes when dissolution tested using United States Pharmacopoeia dissolution methodology (Apparatus 2—paddles@ 50 RPM, 0.1N HCl at 37° C.).

In a particular embodiment, the extended release dosage form of the present invention comprises TPR beads and IR beads; wherein the TPR beads each comprise: an inert bead; an acid layer disposed over the inert bead, comprising the pharmaceutically acceptable organic acid such as fumaric acid; the SR or TPR layer disposed over the acid layer; a drug layer disposed over the SR layer (e.g., comprising ethyl cellulose, optionally plasticized), wherein the drug layer comprises a selective serotonin 5-HT₃ antagonist such as ondansetron (or a salt and/or solvate thereof); and the TPR layer (e.g., comprising ethyl cellulose and hydroxypropyl methylcellulose phthalate, optionally plasticized) is disposed over the drug layer and optionally on the organic acid layer. The IR particles may optionally comprise a granulate of the pharmaceutically acceptable organic acid (e.g. fumaric acid), the selective serotonin 5-HT₃ antagonist (e.g. ondansetron or a salt and/or solvate thereof), and an optional binder (e.g. hydroxypropyl cellulose), as well as one or more additional excipients (e.g. a fillers such as lactose and/or microcrystalline cellulose, a disintegrant such as crospovidone, etc.).

In certain embodiments of the present invention, the extended release dosage form developed in the form of a patient-friendly orally disintegrating tablet is intended for oral administration, once-daily, in patients at moderate to high risk of postoperative nausea and vomiting (PONV) or post discharge nausea and vomiting (PDNV) prior to and/or following inpatient or outpatient ambulatory surgery, and optionally, once-daily up to additional 4 days following the first dose.

In certain other embodiments of the present invention, the extended release dosage form developed in the form of a patient-friendly orally disintegrating tablet is intended for oral administration, once-daily, in cancer patients for the prevention of nausea and vomiting prior to undergoing emetogenic cancer chemotherapy, and optionally, once-daily up to additional 2 days following the first dose.

In yet certain other embodiments of the present invention, the extended release dosage form developed in the form of a patient-friendly orally disintegrating tablet is intended for oral administration, once-daily, in cancer patients for the prevention of nausea and vomiting prior to receiving total body irradiation, single high-dose fraction to the abdomen, or daily fractions to the abdomen, and optionally, once-daily up to additional 2 days following the first dose.

### BRIEF DESCRIPTION OF THE DRAWINGS

FIG. 1.A illustrates the cross-section of an SR coated, organic acid-containing core.

FIG. 1.B illustrates a cross-section of a TPR bead of an antiemetic antagonist comprising an SR coated, organic acid-containing core.

FIG. 2 illustrates the release profiles of both fumaric acid and ondansetron hydrochloride from the TSR beads of Example 1.

FIG. 3 illustrates the release profiles of ondansetron hydrochloride from the TSR beads of Example 2.

FIG. 4 illustrates the ondansetron release profiles from the MR capsule formulations of Example 3 (pilot CTM: PF391EA0001, pivotal CTM: PF392EA0001, and pilot CTM: PF379EA0001).

FIG. 5 illustrates the ondansetron plasma concentration—time profiles of MR capsule formulations (PF391EA0001, PF392EA0001, and PF379EA0001) comprising RR Granules (rapid release granules) and TPR beads of Example 3.

FIG. 6 demonstrates the simulated relationship between (0-24 hr) methods using a once-daily 24 mg dose for post operative nausea and/or vomiting vs. Zofran® 8 mg (line) with 90% prediction intervals (blue colored).

### DETAILED DESCRIPTION OF THE INVENTION

All documents cited herein are incorporated by reference in their entirety for all purposes; the citation of any document is not to be construed as an admission that it is prior art with respect to the present invention.

As used herein, various terms are defined as described in “How to study postoperative nausea and vomiting”, Acta Anaesthesiol. Scand. 2002:46:921-928:
“nausea” refers to a subjective sensation of an urge to vomit, in the absence of expulsive muscular movements; when severe, it is associated with increased salivary secretion, vasomotor disturbances, and sweating;
“vomiting” or “emesis” refers to the forcible expulsion through the mouth of the gastric contents. Vomiting results from coordinated activity of the abdominal, intercostals, laryngeal, and pharyngeal muscles;
“retching” refers to an unproductive effort to vomit, or the rhythmic action of respiratory muscles preceding vomiting;
“incidence” refers to a risk measure associated with developing some new condition within a specified period of time. Incidence=% patients with one or more events wherein an event is nausea, emesis or taking rescue medication;
“incidence rate” refers to the total number of incidence events divided by the duration of the observation interval in which the incidence events occurred, expressed as a rate (e.g., %/hour);
“exposure” refers to the area under the plasma concentration—time profile from time=0 to time=t (e.g., AUC_{0-2 hr}).

Co-pending U.S. patent application Ser. No. 11/668,167 filed Jan. 29, 2007 (Publication No. U.S. 2007/0196491) and U.S. patent application Ser. No. 12/209,285 filed on Mar. 12, 2008 (Publication No. U.S. 2009/0232885), disclose once-daily ondansetron HCl ER Capsules comprising immediate release (IR) beads or rapid-release (RR) granules and timed pulsatile-release (TPR) beads, which provide target plasma profiles suitable for a once-daily regimen as evident in the pilot pharmacokinetic study in healthy volunteers that compared plasma concentration-time profiles of once-daily test formulations with that of Zofran IR tablets orally dosed bid 8 hrs apart. These capsules are designed to be swallowed whole, and thus may be difficult to administer to certain patients having difficulty swallowing.

In a co-pending Provisional patent application Ser. No. 12/688,493, filed on Jan. 14, 2010, entitled “Methods of Treating PDNV and PONV with Extended Release Ondansetron Compositions” a PK/PD model based on ondansetron exposure, AUC_{0-2 hr} (area under plasma concentration curve during the first 2 hours of post-dose) and the corresponding onset and duration of antiemetic responses for Zofran®, an IR ondansetron formulation, with nausea, vomiting, and rescue medication as incidence endpoints was used to compare ondansetron bioavailability for three modified-release formulations of ondansetron and Zofran® (bid). The model shows that oral administration of once-daily MR ondansetron capsules is as effective, if not superior to Zofran® administered bid in preventing nausea and/or vomiting in subjects at moderate to high risk of PONV or PDNV following inpatient or outpatient ambulatory surgery.

As used herein, as well as in specific examples thereof, reference to a drug or drug class (e.g., selective serotonin 5-HT₃ antagonist, ondansetron, etc.) includes the drug itself, as well as pharmaceutically acceptable salts, polymorphs, stereoisomers and mixtures thereof.

As used herein, the term “immediate release” (IR) refers to the release of greater than or equal to about 50%, in some embodiments greater than about 75%, or more than about 90%, and in certain embodiments greater than about 95% of the drug within about 30 minutes when dissolution tested in 0.1N HCl, or within about one hour following administration of the dosage form. Immediate release particles (IR particles) are drug-containing particles which provide immediate release of the drug.

As used herein, the term “immediate-release (IR)” refers to drug-containing particles which release greater than about 50% of the drug within about 30 minutes of dosing. “Rapid release” (RR) refers to drug-containing particles in which at least about 80% of the drug contained in particle is released in about 30 minutes or less for example when dissolution tested using United States Pharmacopoeia (USP) dissolution methodology (Apparatus 2—paddles@ 50 RPM, 0.1N HCl at 37° C., more preferably) and provides a drug release profile similar to that of RLD (reference-listed drug; e.g., the test drug release profile having a similarity factor (f₂) of greater than 50, preferably greater than 75, more preferably greater than 85% when compared to the drug release profile for the corresponding RLD such as Zofran®). For example, RR particles can include, but are not limited to particles in which the drug is layered on 45-60 mesh, or 60-80 mesh sugar spheres, as well as water-soluble microgranules comprising the drug and a filler, (e.g., lactose) and an organic acid (e.g., fumaric acid). Rapid release particles are a particular type of IR particles with relatively high rates of drug release.

The term, “TPR (timed, pulsatile release) bead” or “TPR dosage form”, as defined here, is characterized by an immediate release pulse or a sustained release profile after a pre-determined lag time. The term “lag-time” refers to a time period wherein less than about 10%, more particularly substantially none, of the dose (drug) is released, and a lag-time of from at least about 2 hours up to 10 hours when dissolution is tested by United States Pharmacopoeia (USP) dissolution methodology (Apparatus 2—paddles@ 50 RPM and a two-stage dissolution medium at 37° C. (first 2 hours in 0.1N HCl followed by testing in a buffer at pH 6.8) is achieved by coating typically with a combination of water-insoluble and enteric polymers (e.g., ethylcellulose and hypromellose phthalate). Similarly, a TPR coating or TPR layer refers to a layer, membrane, or coating which provides such properties. As described herein, TPR coatings or layers comprise a pharmaceutically acceptable water insoluble polymer combined with an enteric polymer, optionally plasticized with one or more pharmaceutically acceptable plasticizers. If the drug particles are provided with a barrier coating prior to applying a lag time coating, such beads are more appropriately referred to as timed, sustained release (TSR) beads, as these beads are likely to provide a sustained-release profile following a preset lag time.

The term “SR layer”, “SR coating”, etc. refers to a layer or coating comprising a pharmaceutically acceptable water insoluble polymer, optionally plasticized with one or more pharmaceutically acceptable plasticizers.

The clinical terms “plasma concentration—time profile”, “Cₘₐₓ”, “AUC”, “Tₘₐₓ”, and “elimination half life” have their generally accepted meanings, and hence, are not redefined. Unless indicated otherwise, all percentages and ratios are calculated by weight based on the total composition.

The term “coating weight” refers to the dry weight of a coating as a percentage of the weight of the substrate prior to coating. For example, a 10 mg particle coated with 1 mg coating (dry weight) has a coating weight of 10%.

The terms “similarity factor” and “f₂” refer to a simple measure for the comparison of the drug release profiles of test and reference listed drugs. It is a function of the mean differences in dissolutions and it takes values in the range of from 0 and 100. A convenient critical value of 50 for similarity of dissolution profiles based on mean difference of 10% at all sampling time points (V. P. Shah, Y. Tsang, P. Sathe, J.-P. Liu, In vitro dissolution profile comparison—statistics and analysis of the similarity factor, f₂. Pharmaceutical Research 15, 889-896 (1998).

The present invention is a method of preparing once-daily orally disintegrating tablet formulations comprising a selective serotonin 5-HT₃ antagonist for orally administering prior to the start of moderately emetogenic chemotherapy or radiotherapy, or inpatient or outpatient ambulatory surgery, for the treatment or prevention of nausea and/or vomiting and optionally every 24 hours for an additional 2 to 4 days thereafter. The dosage form comprises TPR particles and IR beads (particularly those with rapid release (RR) characteristics similar to that of a reference listed drug (RLD), an IR product (e.g. Zofran®)), each comprising a selective serotonin 5-HT₃ antagonist (e.g. ondansetron). The TPR particles comprise a core comprising the selective serotonin 5-HT₃ antagonist and a pharmaceutically acceptable organic acid (e.g. fumaric acid) separated from each other by an SR layer comprising a water insoluble polymer (such as ethyl cellulose). The IR particles comprise the selective serotonin 5-HT₃ antagonist, and release at least 80 wt. % of the selective serotonin 5-HT₃ antagonist in about 30 minutes (using USP dissolution methodology (Apparatus 2—paddles @50 RPM in 0.1 N HCl at 37° C.)).

In one embodiment, the oral dosage form for use in the method of the present invention can be prepared as described in copending U.S. patent application Ser. No. 12/209,285, filed Sep. 12, 2008 (Publication No. U.S. 2009/0232885), which is herein incorporated by reference in its entirety for all purposes.

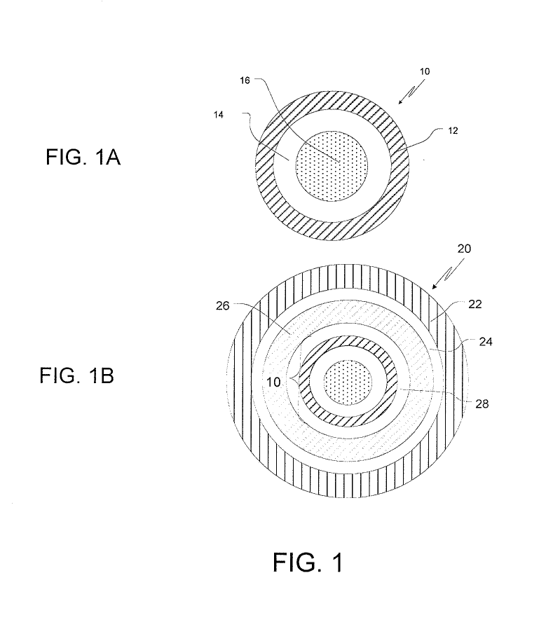

Specific embodiments of the present invention will be described in further detail with reference to the accompanying FIGS. 1.A and 1.B. In FIG. 1.A, an SR-coated core 10 comprising an SR coating 12 applied on an organic acid-containing core comprising a layer of a pharmaceutically acceptable organic acid in a binder 14 coated on an inert particle core 16. The inert particle core 16, organic acid-coating layer 14 and a dissolution rate controlling SR layer 12 make up the SR-coated organic acid-containing core 10. In FIG. 1.B, a representative TPR bead is illustrated. The TPR bead 20 comprises a lag-time coating 22 applied on a primary SR layer 24, a protective seal-coat 26 and a weakly basic drug layer 28 applied on an SR-coated acid-containing core 10. In certain embodiments of the present invention, the intermediate SR barrier layer is not applied, i.e., the TPR layer is directly applied over the seal coated immediate release beads. In some other embodiments of the present invention, the organic acid is simply an organic acid crystal with a desired mean particle size and this crystal is further coated with n SR coating prior to drug layering.

In one embodiment, the pharmaceutical compositions suitable for use in the method of the present invention comprise a plurality of TPR and IR particles, wherein the TPR particles each comprise a core coated with a TPR layer; the core comprises a selective serotonin 5-HT₃ antagonist (e.g. ondansetron) and a pharmaceutically acceptable organic acid separated from each other by an SR layer; and the IR particles each comprise the selective serotonin 5-HT₃ antagonist (e.g. ondansetron) in combination with suitable excipients.

In certain other embodiment, the pharmaceutical compositions suitable for use in the method of the present invention comprise IR particles and one or more TPR bead populations differing in lag time.

In a particular embodiment, the TPR particles comprise an inert core (e.g., a sugar bead etc.) sequentially coated with a pharmaceutically acceptable organic acid (e.g., fumaric acid) and a pharmaceutically acceptable binder (e.g., hydroxypropyl cellulose); a sustained release (SR) layer (e.g., comprising a pharmaceutically acceptable water insoluble polymer such as ethyl cellulose, optionally plasticized with a pharmaceutically acceptable plasticizer such as triethyl citrate or polyethylene glycol); a drug layer comprising the selective serotonin 5-HT₃ antagonist (e.g., ondansetron or a pharmaceutically acceptable salt and/or solvate thereof) and a pharmaceutically acceptable binder (e.g., povidone); an optional sealing layer (e.g. comprising a water soluble polymer such as hydroxypropyl methylcellulose); and a TPR layer (e.g., comprising a water insoluble polymer such as ethyl cellulose, an enteric polymer such as hydroxypropylmethylcellulose phthalate, and an optional pharmaceutically acceptable plasticizer such as triethyl citrate).

In certain embodiments of the present invention, the IR beads/particles release at least about 50% of the selective serotonin 5-HT₃ antagonist within about 30 minutes when dissolution tested in 0.1N HCl, or achieve Cₘₐₓ within about one hour or similar to that of the RLD following administration of the dosage form. In particular embodiments, the IR particles are RR particles, and release at least about 80 wt. % of the selective serotonin 5-HT₃ antagonist in about 30 minutes when dissolution tested using United States Pharmacopoeia (USP) dissolution methodology (Apparatus 2—paddles@ 50 RPM, 0.1N HCl at 37° C.

In a particular embodiment of the present invention, the IR or RR particles can have any suitable structure which provides the required rapid release properties. For example, the IR/RR particles can comprise the selective serotonin 5-HT₃ antagonist deposited directly on an inert core (e.g., 60-80 mesh sugar sphere, cellulose sphere (e.g., Celphere 102 from Asahi Kesahi or Cellets 100 or Cellets 200 from Glatt), cellulose-lactose sphere with a smaller average diameter) and optionally, with a pharmaceutically acceptable hinder. In certain other embodiments to achieve faster release similar to that of Zofran, the RR (IR) particles comprise the selective serotonin 5-HT₃ antagonist disposed over seal coated organic crystals or organic acid-containing cores which are prepared by depositing an organic acid-polymeric binder layer onto inert cores. In contrast IR particles comprising the selective serotonin 5-HT₃ antagonist to be further processed into TPR beads comprise the selective serotonin 5-HT₃ antagonist disposed over an SR or TPR coated organic acid crystals or SR coated organic acid-containing cores which are prepared by depositing an organic acid-polymeric binder layer onto inert cores.

In a particular embodiment, the preferred extended release oral dosage form useful for the prevention of nausea and vomiting is an orally disintegrating tablet (ODT) compressed with required amounts of TPR beads and IR or RR particles, wherein the TPR particles comprise inert cores sequentially coated with an organic acid and a binder (e.g., hydroxypropyl cellulose); a sustained release (SR) layer comprising a water-insoluble polymer (e.g., ethyl cellulose) or a timed, pulsatile release layer comprising a water-insoluble polymer in combination with an enteric polymer (e.g., hypromellose phthalate, HP-55) and an optional plasticizer (e.g., optionally triethyl citrate); a drug layer comprising a elective serotonin 5-HT₃ antagonist and a binder (e.g., povidone); an optional sealing layer (e.g. hydroxypropyl methylcellulose); and a TPR layer comprising ethyl cellulose, hydroxypropylmethylcellulose phthalate, and an optional plasticizer (e.g., optionally triethyl citrate); and the IR/RR particles comprise a serotonin 5-HT₃ antagonist and a binder disposed over an SR-coated or a seal-coated coated organic acid such as fumaric acid, crospovidone, microcrystalline cellulose, and hydroxypropyl cellulose.

A non-limiting list of selective serotonin 5-HT₃ antagonists suitable for use in the extended release compositions include ondansetron, tropisetron, granisetron, dolasetron, palonosetron, ramosetron, and salts and/or solvates thereof. In a particular embodiment, the selective serotonin 5-HT₃ antagonist is ondansetron, or salts and/or solvates thereof.

A non-limiting list of water-insoluble polymers, suitable for use in the TPR and SR layers includes ethylcellulose, cellulose acetate, cellulose acetate butyrate, polyvinyl acetate, neutral copolymers of ethyl acrylate and methylmethacrylate, copolymers of acrylic and methacrylic esters containing quaternary ammonium groups, and waxes. The water-insoluble polymer used in the TPR layer can be the same as or different from the water-insoluble polymer used in the SR layer. In a particular embodiment, the water-insoluble polymer for both the TPR and SR layers is ethylcellulose.

A non-limiting list of enteric polymers suitable for use in the TPR layer includes cellulose acetate phthalate, hydroxypropyl methylcellulose phthalate, hydroxypropyl methylcellulose acetate succinate, polyvinyl acetate phthalate, pH-sensitive copolymers of methacrylic acid and methylmethacrylate, and shellac. In a particular embodiment, the enteric polymer of the TPR layer is hydroxypropyl methylcellulose phthalate.

A non-limiting list of pharmaceutically acceptable organic acids includes citric acid, lactic acid, fumaric acid, malic acid, maleic acid, tartaric acid, succinic acid, oxalic acid, aspartic acid, and glutamic acid. In a particular embodiment, the pharmaceutically acceptable organic acid is fumaric acid.

As discussed and exemplified herein, the IR microparticles can be optionally taste-masked with a taste-masking membrane. The taste-masking membrane can comprise a water-insoluble polymer, which may be unplasticized or plasticized. A non-limiting list of suitable water-insoluble polymers include ethylcellulose, polyvinyl acetate (for example, Kollicoat SR#30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

The taste-masking membrane can further include a gastrosoluble pore former. Representative examples of gastrosoluble organic or inorganic pore-forming agents include, but are not limited to, calcium carbonate, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, and the like and the mixtures thereof. The ratio of water-insoluble polymer to gastrosoluble organic or inorganic pore-former for producing taste-masked particles may typically vary from about 95/5 to about 1/1, or in some embodiments from about 9/1 to 1/1.

In another embodiment, the gastrosoluble pore-former is a polymeric material, for example a terpolymer based on aminoalkyl acrylate or methacrylate, butyl acrylate or methacrylate, and a methacrylate. In another embodiment, the pore-forming polymeric material may be a terpolymer based on dimethylaminoethyl methacrylate, butyl methacrylate, and methyl methacrylate; and in yet another embodiment, the terpolymer has an average molecular weight of 150,000 and the ratio of the monomers is 1:2:1 of methyl methacrylate, N,N-.dimethylaminoethyl methacrylate, and butyl methacrylate. An example of a polymeric gastrosoluble pore-forming material is a polymer of the EUDRAGIT® E series (e.g., EUDRAGIT® E100 or EUDRAGIT® EPO). A polymer of this series has a pKa of 6.3, is soluble in gastric fluid below pH 5 while it swells and/or is permeable in water and buffer solutions above pH 5.0. The saliva is typically in the pH range of about 6.7 to 7.4. Another example of gastrosoluble pore-forming polymer is poly(vinylacetal diethylaminoacetate) e.g., AEA® available from Sankyo Company Limited, Tokyo (Japan). In one embodiment, the gastrosoluble pore-forming polymer is a terpolymer based on dimethylaminoethyl methacrylate, butyl methacrylate, and methyl methacrylate. In another embodiment, the terpolymer has an average molecular weight of 150,000 and the ratio of the monomers is 1:2:1 of methyl methacrylate, N,N-dimethylaminoethyl methacrylate, and butyl methacrylate. The ratio of water-insoluble polymer to pore-forming polymeric material may typically vary from about 95/5 to about 1/1, or about 9/1 to about 1/1.

As discussed herein, the TPR and SR layers can each optionally include a plasticizer. In some cases, it may be desirable to omit a plasticizer (e.g. in order to reduce cost, reduce exposure of patients to plasticizers, etc.). One of skill in the pharmaceutical arts can select suitable grades of water-insoluble polymers and/or enteric polymers amenable to forming a coating without plasticizer. Alternatively, it may be desirable to incorporate a plasticizer into one or both of the TPR and SR layers (e.g. in order to adjust the physical properties of the respective layers, or adjust the release rate of the drug and/or organic acid). When a plasticizer is used, a non-limiting list of suitable plasticizers includes triacetin, tributyl citrate, triethyl citrate, acetyl tri-n-butyl citrate, diethyl phthalate, dibutyl sebacate, polyethylene glycol, polypropylene glycol, castor oil, acetylated mono- and di-glycerides and mixtures thereof. When a plasticizer is used in both the TPR and SR layers, the plasticizer can be the same or different. In one embodiment, the plasticizer of the SR layer is triethyl citrate. In another embodiment, the plasticizer of the TPR layer is triethyl citrate. In yet another embodiment of plasticizer of both the TPR and SR layers is triethyl citrate.

As described herein, the method of making an orally disintegrating tablet of the present invention further comprises the process of making rapidly dispersing microgranules comprising at least one super disintegrant and at least one sugar alcohol or one sugar saccharide at a ratio of 10:90 to 1:99 using purified water by high shear granulation followed by fluid-bed drying or dry in a tray drying oven.

In one embodiment, the TPR particles comprise “layered beads” in which the organic acid and drug are layered onto an inert core. The inert core can be any pharmaceutically acceptable inert core; in particular those with an average particle size of 50-400 μm. A non-limiting list of suitable inert cores includes sugar spheres, cellulose spheres, lactose spheres, lactose-MCC spheres, mannitol-MCC spheres, and silicon dioxide spheres.

Antiemetic drugs such as domperidone, granisetron, cyclizine, droperidol, dexamethasone, and ondansetron, as well as combinations of these drugs have been used to treat postoperative nausea and vomiting (PONV) or postdischarge nausea and vomiting (PDNV), or to prevent nausea and vomiting undergoing moderately emetogenic cancer chemotherapy or radiotherapy.

The ODTs of the present invention may be administered to a patient in need thereof, or may be administered in combination with an oral dosage form comprising another type of antiemetic drug. For example, the method of combination treatment comprising treating or preventing PONV and/or PDNV by administering at least one extended release ODT dosage form comprising a selective serotonin 5-HT₃ antagonist to a surgical patient in need thereof, in most embodiments prior to, in some after surgery, or at discharge, and further administering at least one additional oral antiemetic comprising one or more NK-1 antagonist, dopamine antagonist, H₁ histamine receptor antagonist, cannabinoid, benzodiazepine, anticholinergic, steroid, etc, preferably in a patient-friendly orally disintegrating tablet form. The coadministration of the extended release dosage form comprising a selective serotonin 5-HT₃ antagonist in the additional oral antiemetic dosage form can include administration of the two dosage forms more or less simultaneously; or at different times, such that clinically significant plasma levels of the selective serotonin 5-HT₃ antagonist and the additional oral antiemetic are present in the patient at more or less the same or different time periods.

In methods of combination treatment as described above, in which an extended release dosage form comprising a selective serotonin 5-HT₃ antagonist is coadministered with an additional oral antiemetic, the NK-1 antagonist can include aprepitant or casopitant; the dopamine antagonist can include domperidone, droperidol, haloperidol, chlorpromazine, or prochlorperazine; the H₁ histamine receptor antagonist can include cyclizine, diphenhydramine, dimenhydrinate, meclizine, promethazine, or hydroxyzine; the cannabinoid can include cannabis, dronabinol, or nabilone; the bezodiazepine can include midazolam or lorazepam; the anticholinergic can be scopalamine; and the steroid can be dexamethasone.

In methods of combination treatment, the extended release ODT dosage form can be administered prior to surgery, immediately after surgery, or at discharge, or can be used in combination with prophylactic administration of an IV antiemetic administered before, during, immediately after surgery, or at discharge. For example, the extended release dosage form can be administered prior to surgery instead of the prophylactic IV antiemetic, thereby providing an effective prophylactic dose of selective serotonin 5-HT₃ antagonist which provides protection against PONV/PDNV immediately after surgery, at discharge, as well as for an extended postoperative period, thereby providing enhanced patient compliance and quality of life, and also reduce medical costs.

In one embodiment, the method of the present invention, as described herein, can be used to treat patients at moderate to high risk to PONV or PDNV undergoing inpatient or outpatient surgical procedures. For example, although intravenous administration is more readily available for inpatient procedures, the present method of administering an extended release dosage form comprising a selective serotonin 5-HT₃ antagonist avoids the risks and expense associated with intravenous administration. For outpatient surgical procedures, it is generally difficult to administer antiemetics intravenously after discharge, and accordingly administration of an oral dosage form is substantially more convenient and less costly. In addition, the present method of administering an extended release ODT dosage form comprising a selective serotonin 5-HT₃ antagonist is a substantial improvement over the currently available immediate release dosage forms, because immediate release dosage forms require multiple daily administrations in order to provide continuous treatment or prophylaxis of PDNV, whereas the present method provides for once-daily administration, resulting in improved compliance and reduced incidence of PDNV. Thus, for example, the extended release dosage form comprising a selective serotonin 5-HT₃ antagonist described herein can be administered immediately prior to discharge and/or once-daily subsequent to discharge (e.g., beginning about 24 hours after discharge, for example in the morning following discharge) for up to one week (for example up to 5 days after discharge) to treat or ameliorate PONY and/or PDNV.

In some embodiments, the extended release ODT dosage form comprising a selective serotonin 5-HT₃ antagonist is effective for prophylaxis or treatment of PONV/PDNV for surgical patients administered postoperative opioids for analgesia. Such opioids can include, for example, codeine, morphine, thebaine, oripavine, diacetyl morphine, dihydrocodeine, hydrocodone, hydromorphone, nicomorphine, oxycodone, oxymorphone, fentanyl, α-methyl fentanyl, alfentanil, sufentanil, remifentanil, meperidine, buprenorphine, etorphine, methadone, and tramadol.

The method of the present invention can be used generally for both cancer patients undergoing moderately emetogenic cancer chemotherapy or total body radiotherapy.

### EXAMPLES

### Example 1

1.A SR-Coated Fumaric Acid Crystals: 40-80 mesh fumaric acid crystals (3750 g) were charged into a Glatt GPCG 5 fluid-bed coater equipped with a 9″ bottom spray Wurster insert, 10″ column length and 16 mm tubing. The fumaric acid crystals were coated with a solution (6% solids) of 250 g of ethylcellulose (EC-10: Ethocel Premium 10 cps) and 166.7 g of polyethylene glycol (PEG 400) at an EC-10/PEG 400 ratio of 60/40, dissolved in 98/2 acetone/water (6528.3 g), for a coating weight of up to 10% by weight. The processing conditions were as follows: atomization air pressure: 2.0 bar; nozzle diameter: 1.00 mm; bottom distribution plate: B with 15 gauge 100 mesh screen; spray/shake interval: 30 s/3 s; product temperature maintained at 35±1° C.; inlet air volume: 155-175 cubic feet per minute (cfm) and a spray rate increasing from about 8 to 30 g/min.

Fumaric acid crystals were also coated as described above using different ratios of ethylcellulose and PEG. More specifically, fumaric acid crystals were coated with a solution of EC-10/PEG 400 at a ratio of either 75/25 or 67.5/32.5, providing a coating weight of up to 10% by weight for each ratio.
1.B Ondansetron Hydrochloride IR Beads: Povidone (PVP K-29/32; 23 g) was slowly added to 50/50 water/Denatured Alcohol 3C, 190 Proof (3699.4 g), with mixing until dissolved. Ondansetron hydrochloride dihydrate (197.2 g) was slowly added to the povidone binder solution until the ondansetron hydrochloride was dissolved. SR-coated fumaric acid crystals (3000 g) obtained from Example 1.A, above, were coated in the Glatt GPCG 5 with the ondansetron solution (5% solids) while maintaining the product temperature at 40±1° C.; inlet air volume at 180-195 cfm and with a spray rate increasing from about 8 to 15 g/min. The resulting drug-layered beads were provided with a protective seal-coat of Opadry Clear (hypromellose 2910; 3 cps) (2% coating weight) to form IR beads.
1.C Ondansetron Hydrochloride TPR Beads: Ondansetron hydrochloride IR beads (2800 g) from Example 1.B were coated by spraying a solution in 98/2 acetone/water (6% solids) of EC-10/hydroxypropylmethyl cellulose (HPMCP; HP-55)/triethyl citrate (TEC) at a ratio of 45.5/40/14.5, and dried in the Glatt for about 10 minutes at 60° C. to drive off excess residual solvent, to provide a coating weight of up to 50%. The dried beads were sieved to discard any “doubles” formed.

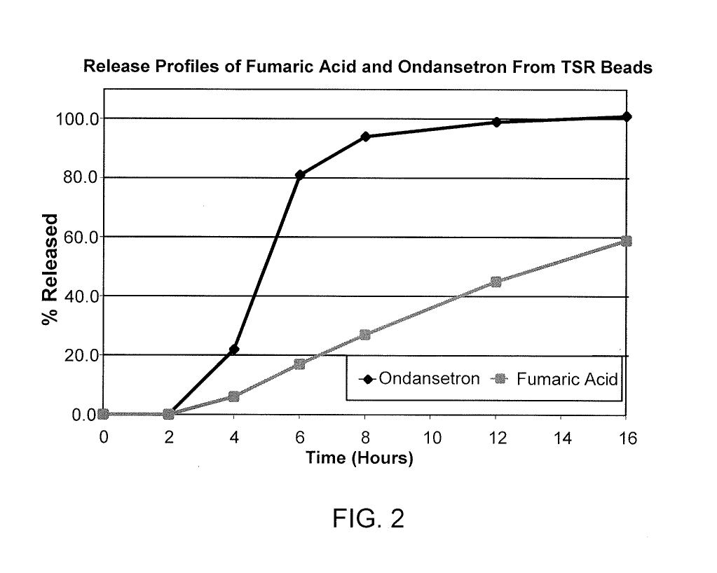

FIG. 2 shows the release profiles of both fumaric acid and ondansetron from TPR beads comprising SR-coated acid crystals. More specifically, the TPR Beads evaluated in FIG. 2 have the following composition:

| | | Coating Weight |
|---|---|---|
| | Composition | (wt. %) |
| core | fumaric acid crystals | N/A |
| SR Layer 1 | EC-10/PEG 400 (60/40) | 10 |
| or | or | or |
| SR Layer 2 | EC-10/PEG 400 (67.5/32.5) | 10 |
| or | or | or |
| SR Layer 3 | EC-10/PEG 400 (75/25) | 10 |
| Drug Layer | ondansetron HCl/PVP (90/10) | 6 |
| TPR Layer | EC-10/HP-55/TEC (45.5/40/14.5) | 50 |

Although the ondansetron release is significantly faster than the fumaric acid release, it will be apparent to a person skilled in the art that by decreasing the thickness of the barrier-coat (SR layer) on the fumaric acid crystals and/or additionally applying a SR layer under the TPR layer to sustain the drug release, the release profiles for both ondansetron and fumaric acid can be synchronized.

### Example 2

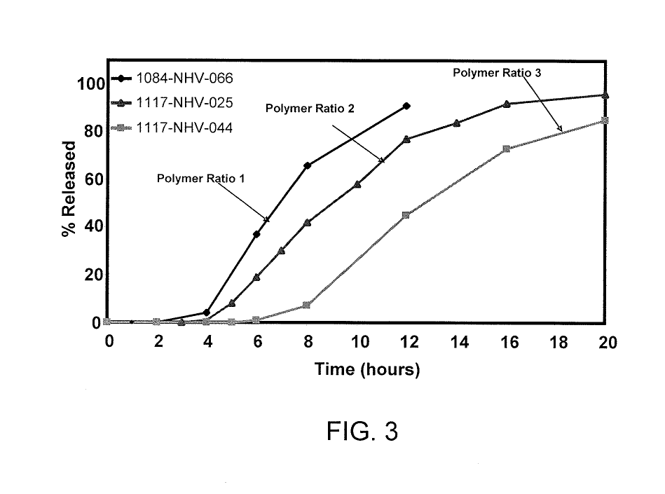

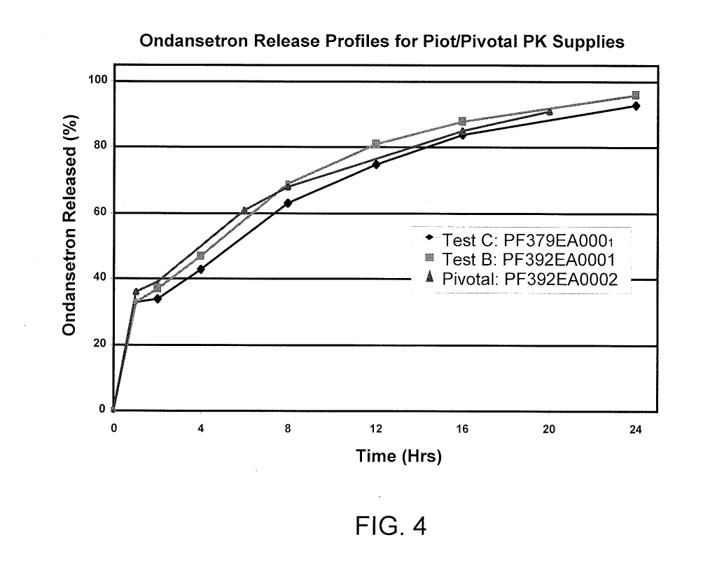

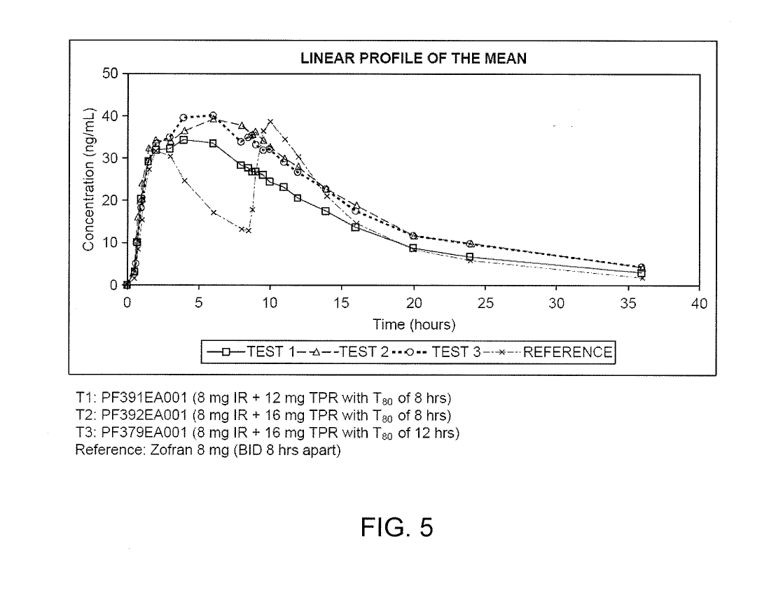

2.A Fumaric Acid-Containing Cores: Hydroxypropyl cellulose (Klucel LF, 53.6 g) was slowly added to 90/10 190 proof alcohol/water at 4% solids, with rigorous stirring until dissolved, and then fumaric acid (482.1 g) was slowly added and stirred until dissolved. A Glatt GPCG 5 equipped with a 9″ bottom spray Wurster insert, 10″ partition column was charged with 3750 g of 25-30 mesh sugar spheres. The sugar spheres were layered with the fumaric acid solution while maintaining the product temperature at about 33-35° C. and at a spray rate of 8-60 mL/min. The acid cores were dried in the Glatt unit for 10 min to drive off residual solvent/moisture and sieved through 40-80 mesh screens.
2.B SR-coated Fumaric Acid-Containing Cores: Following the procedures of Example 1.A, fumaric acid cores (3750 g) from Example 2.A were coated with a solution of EC-10 mixed with either PEG 400 (B.1) at a ratio of 60/40 or TEC (B.2) at a ratio of 90/10 as the plasticizer, dissolved in 98/2 acetone/water (6% solids), providing a coating weight of 10%.
2.C Ondansetron Hydrochloride IR Beads: Ondansetron hydrochloride IR beads (B.1 and B.2) were prepared as described in Example 1.B by coating the SR-coated fumaric acid-containing cores of Example 2.B with a solution of ondansetron hydrochloride dihydrate/povidone (90/10) at a drug load of 4 wt. % (as ondansetron base). The resulting drug-layered beads were provided with a protective seal-coat with Pharmacoat 603 (hypromellose 2910; 3 cps) for a weight gain of 2%.
2.D Ondansetron Hydrochloride SR Beads: Ondansetron hydrochloride IR beads (1080 g) from Example 2.0 were SR coated by spraying a solution of EC-10 mixed with either PEG 400 (D.1) at a ratio of 60/40 or TEC (D.2) as the plasticizer at a ratio of 90/10, dissolved in 98/2 acetone/water (7.5% solids), and dried in the Glatt at the same temperature for 10 minutes to drive off excess residual solvent, providing a coating weight of 10%. The dried beads were sieved to discard any doubles, if formed.
2.E Ondansetron Hydrochloride TSR Beads: Ondansetron hydrochloride SR beads (D.1 and D.2) from Example 2.D, were further coated with a TPR coating of EC-10/HP-55/TEC at three ratios: 45.5/40/14.5 (E.1—lot #1084-066), 50.5/35/14.5 (E.2—lot #1117-025) and 60.5/25/14.5 (E.3—lot #1117-044) dissolved in 90/10 acetone/water (7.5% solids), at coating weights of up to 50%. The resulting TSR beads were dried in the Glatt to drive off residual solvent and sieved through a 18 mesh screen. FIG. 3 shows the release profiles of ondansetron hydrochloride from TSR beads coated with EC-10/HP-55/TEC at three different ratios (E.1, E.2 and E.3). More specifically, FIG. 3 shows the release profiles for the following formulations:

| | | |
|---|---|---|
| | | Coating Weight |
| Description | Composition | (%) |

E.1: Lot# 1084-066

| | | |
|---|---|---|
| Core | 25-30 mesh sugar spheres | N/A |
| Acid Layer | fumaric acid/Klucel (90/10) | 6.0 |
| SR Layer | EC-10/PEG 400 (60/40) | 10 |
| Drug Layer | ondansetron HCl/PVP (90/10) | 5 (no seal coat) |
| (4% ondansetron | | |
| base) | | |
| SR Layer | EC-10/TEC (90/10) | 10 |
| TPR Layer | EC-10/HP-55/TEC (45.5/40/14.5) | 50 |

E.2: Lot# 1117-025

| | | |
|---|---|---|
| Core | 25-30 mesh sugar spheres | N/A |
| Acid Layer | fumaric acid/Klucel (90/10) | 6 |
| SR Layer | EC-10/TEC (90/10) | 10 |
| Drug Layer | ondansetron/Klucel LF (90/10) | 7 |
| (4% ondansetron | | |
| base) | | |
| SR Layer | EC-10/TEC (90/10) | 10 |
| TPR Layer | EC-10/HP-55/TEC (50.5/35/14.5) | 50 |

E.3: Lot# 1117-044

| | | |
|---|---|---|
| Core | 25-30 mesh sugar spheres | N/A |
| Acid Layer | fumaric acid/PVP (90/10) | 6 |
| SR Layer | EC-10/TEC (90/10) | 10 |
| Drug Layer | ondansetron/Klucel LF (90/10) | 7 |
| (4% ondansetron | | |
| base) | | |
| SR Layer | EC-10/TEC (90/10) | 10 |
| TPR Layer | EC-10/HP-55/TEC (60.5/25/14.5) | 50 |

### Example 3

3.A Ondansetron Hydrochloride IR Beads at a drug load of 10%: Hydroxypropylcellulose (Klucel LF from Aqualon, 33 g) was slowly added to 50/50 water/Denatured Alcohol 3C, 190 Proof (2500 g each) while mixing to dissolve. Ondansetron hydrochloride dihydrate (300 g) was slowly added to the above binder solution until the drug was dissolved. 60-80 mesh sugar spheres (2607 g) were coated with the drug solution (5% solids) in a Glatt GPCG 5 to provide a drug content of 10 wt. % (as ondansetron base) under the following conditions: air distribution plate: B with 100 mesh screen; nozzle diameter: 1 mm; partition height: 10″; 9″ bottom spray Wurster insert; product temperature at 36-37° C.; inlet air volume at 60-65 cfm and increasing spray rate from about 20-25 g/min. The resulting drug-layered beads were provided with a protective seal-coat of Pharmacoat 603 (hypromellose 2910; 3 cps) (2% weight gain) to form IR beads. The IR beads were dried in the Glatt unit for 10 min to drive off residual solvent/moisture and sieved through 40-80 mesh screens. More than 90% of the IR beads were in the particle size range of 100-350 μm.
3.B Ondansetron Hydrochloride RR Granules at a drug load of 10%: Fumaric acid (270 g), Klucel LF (120 g), and ondansetron HCl (600 g) were slowly added to a 50/50 mixture of Denatured 190 Proof Ethyl Alcohol and water (5000 g each) in a stainless steel tank, with agitation until dissolved. A Glatt GPCG 5 equipped with a top spray Wurster insert was pre-heated for not less than 30 min and charged with spray dried lactose (Fast Flo Lactose; 2130 g), microcrystalline cellulose (MCC, Avicel PH102; 2400 g); Crospovidone (XL-10; 480 g), which were then granulated while spraying with the ondansetron solution at a rate of 25-100 g/min under the following conditions: granulating bowl: GPCG 5 with top spray; nozzle tip: 1.2 mm; inlet air temperature: 55° C.; air flow target: 80 cfm; atomization air pressure: 2.0 bar; product temperature target: 50° C. The granulation was dried at 55° C. to a loss on drying (LoD) value of <2%. The granules were sieved through a 20 mesh screen and blended with magnesium stearate (10 g per 5000 g of granules) in a 0.5 cu.ft. V blender rotating at 21 RPM for 5 minutes.
3.C Fumaric Acid-Containing Cores: 25-30 mesh sugar spheres (3750 g) were layered with fumaric acid (482.1 g) from a solution (4% solids) of Klucel LF (53.6 g) as described in Example 2.A above, to achieve a fumaric acid load of 11.25% by weight. The fumaric acid-containing cores were dried in the Glatt unit for 10 min to drive off residual solvent/moisture, and sieved through 20-30 mesh screens.
3.D SR-coated Fumaric Acid Cores: The fumaric acid-containing cores (3750 g) from Example 3.C were coated with a solution of 177.6 g of ethylcellulose (EC-10) and 19.7 g of triethyl citrate (TEC) at a ratio of 90/10, dissolved in 95/5 acetone/water (7.5% solids), providing a coating weight of 5%.
3.E Ondansetron Hydrochloride IR Beads: IR beads of ondansetron hydrochloride dihydrate with a drug load of 10% by weight were produced by spraying a solution (5% solids) of ondansetron hydrochloride dihydrate (402.8 g) and Klucel LF (44.3 g) dissolved in a 50/50 ethanol/water mixture (4247.4 g each), onto SR-coated fumaric acid cores (3500 g) from Example 3.D, above, in a Glatt GPCG 5 under the following conditions: air distribution plate: B with 15 gauge 100 mesh screen; nozzle diameter: 1 mm; partition height: 10″; 9″ bottom spray Wurster insert; product temperature at 34±1° C.; inlet air volume at 150 cfm; atomization air pressure—1.5 bar; and an increasing spray rate of from 8 to 30 mL/min. The resulting drug-layered beads were provided with a protective seal-coat of Pharmacoat 603 (hypromellose 2910; 3 cps) (2% weight gain) to form IR beads with an ondansetron content of 10 wt. % (as ondansetron base). The resulting IR beads were dried in the Glatt unit for 10 min to drive off residual solvent/moisture, and sieved to discard oversized and undersized particles.
3.F-1 Ondansetron Hydrochloride TPR Beads at 15% Coating: Ondansetron hydrochloride IR beads (3500 g) from Example 3.E, above, were coated with a TPR coating of ethylcellulose (389.1 g), HP-55 (135.9 g) and TEC (92.6 g) (ratio: 63/22/15) dissolved in 90/10 acetone/water by spraying the solution (18% solids) to a coating weight of 15%, and dried in the Glatt at the coating temperature for 10 minutes to drive off excess residual solvent. The dried beads were sieved to discard any doubles, if formed.
3.F-2 Ondansetron Hydrochloride TPR Beads at 10% Coating: Ondansetron hydrochloride IR beads (3500 g) from Example 3.E, above, were coated with a TPR coating of ethylcellulose (245.0 g), HP-55 (85.6 g) and TEC (58.3 g) (ratio: 63/22/15) dissolved in 90/10 acetone/water by spraying the solution (18% solids) to a coating weight of 10%, and dried in the Glatt at the coating temperature for 10 minutes to drive off excess residual solvent. The dried beads were sieved to discard any doubles, if formed.

### Example 4

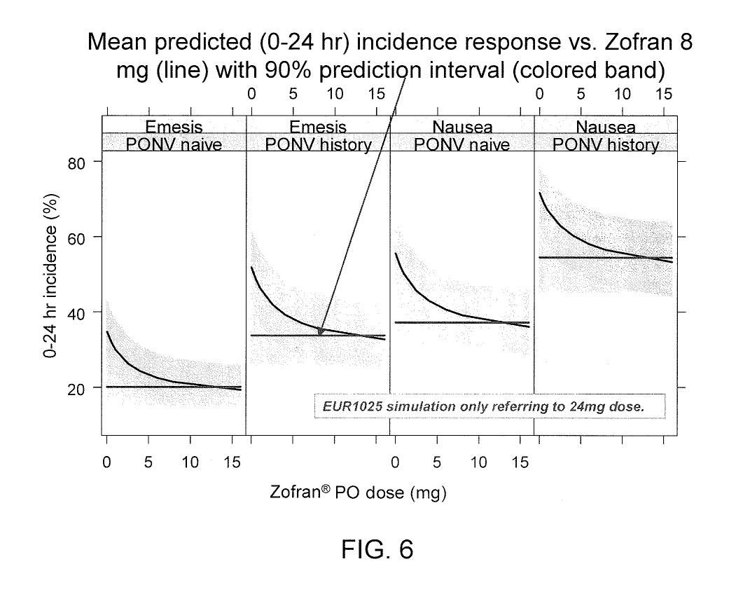

4.A Ondansetron IR Beads: A Glatt GPCG 3 equipped with a bottom spray Wurster insert is pre-heated to a process air temperature of 36° C. and air volume of 150 cfm and charged with 60-100 mesh aspartic acid crystals (1.60 kg) and coated with a TPR coating solution comprising ethylcellulose, HP-55 and TEC at a ratio of 60/30/10 dissolved in 90/10 acetone/water (6% solids) while spraying at a rate of 6-15 g/min under the following conditions: top spray nozzle tip: 1.8 mm; inlet air temperature: 60-70° C.; air flow target: 150-200 cfm; atomization air pressure: 2.0 bar; product temperature target: 31-34° C. Following completion of the coating for a weight gain of 30%, the TPR coated beads are layered with ondansetron from a polymer binder solution following the procedures of Ex. 2.C above for a drug load of 15 wt. % (as free base) based on the weight of IR beads that includes a 2% protective seal coating layer disposed over drug-layered beads. The IR beads are dried in the same unit to minimize residual solvent levels and sieved to discard doubles and fines, if any.
4.B Ondansetron TPR Beads: Ondansetron hydrochloride IR beads prepared in Ex. 4.A above are sprayed with a TPR coating solution (6% solids; 65/25/10 ethylcellulose/HP-55/TEC) for a weight gain of 40 wt. % as disclosed above, then dried in the Glatt at a process air temperature of 45° C. and an air volume of 500 cfm for 10 minutes to drive off excess residual solvent. The dried beads are sieved to discard doubles, if formed.
4.C Ondansetron Taste-masked Beads: Ondansetron IR beads prepared above are taste-masked by coating in a fluid bed coater (e.g., a Glatt GPCG 3) with a solution of Ethocel 10 cps and Eudragit EPO at a ratio of 50:50 in accordance with the disclosures of co-pending patent application Ser. No. 11/248,596 filed Oct. 12 (Publication No. 2006/0078614), 2005 for a weight gain of 20%. The taste masked beads are dried in the unit for 10 min to drive off residual solvent/moisture and sieved through 40-80 mesh screens.
4.D Rapidly-dispersible microgranules: The rapidly-dispersing microgranules comprising a sugar alcohol such as mannitol and a disintegrant such as crospovidone are prepared following the procedure disclosed in the co-pending U.S. patent application Ser. No. 10/827,106, filed Apr. 19, 2004 (Publication No. U.S. 2005/0232988), the contents of which are hereby incorporated by reference. D-mannitol (152 kg) with an average particle size of approximately 20 μm or less (Pearlitol 25 from Roquette, France) is blended with 8 kg of cross-linked povidone (Crospovidone XL-10 from ISP) in a high shear granulator (GMX 600 from Vector) and granulated with purified water (approximately 32 kg) and wet-milled using a rotary mill from Quadro and dried in a Greunburg oven. The rapidly-dispersing microgranules thus obtained will have an average particle size in the range of approximately 20-300 μm.
4.E Ondansetron Hydrochloride ODT CR, 24 mg: Rapidly-dispersing microgranules (5402 g) are blended with taste masked IR beads (695 g), TPR beads at 30% coating (1778 g), and pre-blended excipient mixture of a flavor, a sweetener, and additional disintegrant (1485 g), in a twin shell V-blender for 15 minutes to get homogeneously distributed blend for compression. Tablets weighing approximately 900 mg are compressed using a production scale tablet press equipped with an external lubrication system at a mean hardness in the range of about 40-50 N and friability of about <0.5% by weight. Ondansetron Hydrochloride Dihydrate MR ODT, 24 mg (as free base) thus produced rapidly disintegrates in the oral cavity creating a smooth, easy-to-swallow suspension comprising coated ondansetron hydrochloride beads, which will provide a target profile suitable for a once-daily dosing regimen.
4.F Pilot/Pivotal Bioequivalence Studies: As per regulatory requirements, a single dose (qd 24 mg) food effect study of ODT CR of the present invention, and a comparative PK study of inventive ODT CR formulation (qd 24 mg) versus ER Capsules (qd, 24 mg) disclosed in U.S. Provisional patent application Ser. No. 12/688,493 filed on Jan. 14, 2010 in adequate number of healthy subjects are conducted, and additional pharmacokinetic data are collected according to the specific pilot/pivotal clinical protocols pre-approved by institutional medical review board. The safety profile of each treatment is also assessed by recording the nature, severity, frequency, duration and relation to the treatment of any adverse event. Pharmacokinetic simulations based on the relationships between the mean incidence-exposure and AUC_{0-t hr} of Zofran as disclosed in the above application were performed and the model-predicted incidence for a 24 mg ODT CR given pre-operatively when overlaid as shown in FIG. 6 should behave similarly.

1. An ODT comprising:
at least one population of taste-masked immediate-release (IR) drug microparticles comprising a selective serotonin 5-HT₃ blocking agent, wherein the taste-masked IR microparticles provide dissolution profiles similar to that of a reference drug product;
at least one population of timed, pulsatile release (TPR) beads comprising the selective serotonin 5-HT₃ blocking agent and at least one pharmaceutically acceptable organic acid, wherein the organic acid is not depleted from the TPR beads until completion of the drug release from the dosage form when dissolution tested by United States Pharmacopoeia (USP) dissolution methodology (Apparatus 2—paddles@ 50 RPM and a two-stage dissolution medium at 37° C. (first 2 hours in 0.1N HCl followed by testing in a buffer at pH 6.8); and
rapidly dispersing microgranules comprising at least one super disintegrant and at least one sugar alcohol, a saccharide, or a mixture thereof

2. The ODT of claim 1, wherein the rapidly dispersing microgranules and the taste-masked IR microparticles and TPR beads have a mean particle size of less than about 400 μm.

3. The ODT of claim 1 wherein:
the taste-masked IR microparticles have an average particle size of less than 400 μm, and are taste-masked with a taste-masking membrane comprising a water-insoluble polymer;
the TPR beads comprise an outer lag-time coating comprising a water-insoluble polymer in combination with an enteric polymer disposed over immediate release selective serotonin 5-HT₃ blocking agent-containing beads, said outer lag-time coating providing a lag time of from about 2 to about 4 hours before onset of drug release when dissolution tested by United States Pharmacopoeia (USP) dissolution methodology (Apparatus 2—paddles@ 50 RPM and a two-stage dissolution medium at 37° C. (first 2 hours in 0.1N HCl followed by testing in a buffer at pH 6.8);
wherein the immediate release selective serotonin 5-HT₃ blocking agent-containing beads comprise at least one selective serotonin 5-HT₃ blocking agent disposed over an SR- or TPR-coated organic acid core particle;
said SR- or TPR-coated organic acid core particle comprises an inner barrier coating disposed over an organic acid core particle, said inner barrier coating comprising a water-insoluble polymer alone or in combination with a water-soluble or enterosoluble polymer and providing a sustained-release profile of said organic acid; and
said organic acid core particle comprises at least one pharmaceutically acceptable organic acid.

4. The ODT of claim 3, wherein the taste-masking membrane disposed on the IR microparticle further comprises a gastrosoluble pore-forming agent, wherein the ratio of water-insoluble polymer to gastrosoluble pore-forming agent varies from about 9:1 to 1:1.

5. The ODT of claim 4, wherein the gastrosoluble pore-forming agent is selected from the group consisting of calcium carbonate, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, and mixtures thereof

6. The ODT of claim 4, wherein the taste-masking membrane comprises water-insoluble ethylcellulose and a gastrosoluble polymer at a ratio of from about 9:1 to 1:1, and is disposed on the IR microparticle at a coating weight of from about 5% to 40% based on the weight of the taste-masked IR microparticle.

7. The ODT of claim 1, comprising rapidly-dispersing microgranules with an average particle size of not more than 400 μm, wherein the super disintegrant and sugar alcohol or saccharide or combination thereof, each have an average particle size of not more than about 30 μm;
wherein the ODT exhibits the following properties:
a friability of less than 1% by weight; and
a disintegration time of about 60 seconds or less on contact with the saliva in the oral cavity, wherein after disintegration in the oral cavity, the ODT forms a smooth suspension.

8. The ODT of claim 1, wherein the ratio of rapidly dispersing microgranules to the total amount of taste-masked IR microparticles and TPR beads ranges from about 4:1 to 1:1, thereby providing a smooth mouthfeel in patients in need of said medication.

9. The ODT of claim 1, wherein the ODT rapidly disintegrates on contact with the saliva in the oral cavity of a patient, thereby forming a smooth (non-gritty), easy-to-swallow suspension comprising taste-masked IR microparticles and TPR beads that are easily swallowed by patients with or without having difficulty in swallowing tablets or capsules.

10. The ODT of claim 1, wherein the ratio of the selective serotonin 5-HT₃ blocking agent in the taste-masked IR microparticles to the total amount of selective serotonin 5-HT₃ blocking agent in the TPR beads varies from about 20:80 to about 60:40.

11. The ODT of claim 1, further comprising at least one water soluble excipient;
wherein the taste-masked IR drug particles comprise an inert core selected from the group consisting of a sugar sphere, cellulosic sphere, cellulose-lactose, cellulose-mannitol, and fused silicon dioxide spheres or granules, layered with the selective serotonin 5-HT₃ blocking agent, and coated with a taste-masking membrane comprising said drug; and
wherein the ratio of the selective serotonin 5-HT₃ blocking agent in the taste-masked IR microparticles to the total amount of selective serotonin blocking agent in the TPR beads varies from about 20:80 to about 60:40.

12. The ODT of claim 1, wherein said weakly basic selective serotonin 5-HT₃ blocking agent is selected from the group consisting of ondansetron, tropisetron, granisetron, dolasetron, palonosetron, ramosetron, and pharmaceutically acceptable salts and/or solvates thereof.

13. The ODT of claim 1, wherein the organic acid is selected from the group consisting of citric acid, fumaric acid, malic acid, maleic acid, tartaric acid, succinic acid, oxalic acid, aspartic acid, glutamic acid and mixtures thereof.

14. The ODT of claim 1, wherein the ratio of selective serotonin 5-HT₃ blocking agent to organic acid varies from about 5:1 to 1:10 by weight, and provides a pharmacokinetic profile suitable for a once-daily dosing regimen.

15. The ODT of claim 1, wherein the selective serotonin 5-HT₃ blocking agent is ondansetron or a pharmaceutically acceptable salt and/or a solvate thereof and the organic acid is fumaric acid.

16. The ODT of claim 1, wherein the TPR beads comprise an organic acid core particle coated with the selective serotonin 5-HT₃ blocking agent;
wherein the organic acid core particle is selected from the group consisting of:
an organic acid crystal;
an inert particle coated with an organic acid and a polymeric binder; and
a pellet containing the organic acid, a polymeric binder and a diluent/filler, prepared by controlled spheronization using a Vector Granurex X-35 or equivalents thereof, a rotogranulator from Glatt or equivalents thereof, or by granulation-extrusion-spheronization.

17. The ODT of claim 1, wherein the TPR beads comprise an organic acid core particle comprising the organic acid, a barrier coating disposed over the organic acid, and the selective serotonin 5-HT₃ blocking agent disposed over the barrier coating;
wherein the barrier coating comprises a water-insoluble polymer optionally in combination with a water-soluble or enteric polymer at a ratio of from about 9:1 to 5:5, and wherein the barrier coating is applied at a coating weight of from about 5% to 40% based on the weight of the TPR bead; and
further comprising an outer lag-time coating disposed over the selective serotonin 5-HT₃ blocking agent, wherein the outer lag-time coating comprises a water-insoluble polymer in combination with an enteric polymer.

18. The ODT of claim 17, wherein the barrier coating comprises the combination of a water-insoluble polymer and a water-soluble polymer selected from the group consisting of methylcellulose, hydroxypropyl methylcellulose, hydroxypropylcellulose, polyvinyl pyrrolidone and polyethylene glycol and mixtures thereof.

19. The ODT of claim 3, 4, or 17, wherein the taste-masking membrane comprises a water-insoluble polymer selected from the group consisting of ethylcellulose, cellulose acetate, cellulose acetate butyrate, polyvinyl acetate, neutral methacrylic acid-methylmethacrylate copolymers, and mixtures thereof.

20. The ODT of claim 3 or 17, wherein the enteric polymer of the TPR beads is selected from the group consisting of cellulose acetate phthalate, hydroxypropyl methylcellulose phthalate, hydroxypropyl methylcellulose succinate, polyvinyl acetate phthalate, pH-sensitive methacrylic acid-methylmethacrylate copolymers, shellac, derivatives thereof, and mixtures thereof.

21. The ODT of claim 16, wherein the organic acid core particle is an inert particle coated with an aorganic acid and a polymeric binder, wherein the polymeric binder is selected from the group consisting of methylcellulose, hydroxypropyl methylcellulose, hydroxypropylcellulose, polyvinyl pyrrolidone and polyethylene glycol and mixtures thereof.

22. The ODT of claim 3, wherein the lag-time coating of the TPR beads comprises water-insoluble ethylcellulose in combination with enteric hypromellose phthalate at a ratio of from about 9:1 to 1:3, respectively, for a coating weight of from about 10% to 60% based on the weight of the TPR beads.

23. The ODT of claim 3, wherein one or more of said taste-masking membrane, said inner barrier coating, and said lag-time coating independently further comprises a plasticizer selected from the group consisting of triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof.

24. The ODT of claim 1, wherein said IR microparticles provide a loading dose by releasing not less than about 85% of the selective serotonin 5-HT₃ blocking agent contained in the taste-masked IR microparticles within 30 minutes under in vitro dissolution conditions.

25. The ODT of claim 1, wherein the composition substantially disintegrates within about 60 seconds after administration in the oral cavity of the patient.

26. The ODT of claim 1, wherein said composition disintegrates within about 30 seconds when tested by the <USP 701> Disintegration Test.

27. The ODT of claim 1, further comprising other pharmaceutically acceptable excipients selected from the group consisting of a flavor, a sweetener, additional disintegrant and a diluent, and the ODT disintegrates in the oral cavity in about 60 seconds creating a smooth, easy-to-swallow suspension of multi-coated beads which provide target pharmacokinetics profiles suitable for a once-daily dosing regimen in patients in need of such a medication.

28. The ODT composition of claim 1, wherein said taste-masked IR microparticles and said TPR beads further comprise a compressible coating applied on individual drug particles to eliminate/minimize potential fracture of the coating membrane during compression into ODT tablets.

29. A method for the preparation of a multiparticulate ODT tablet comprising a selective serotonin 5-HT₃ blocking agent, and at least one pharmaceutically acceptable organic acid, comprising the steps of:
preparing rapidly releasing selective serotonin 5-HT₃ blocking agent-containing immediate release (IR) microparticles;
applying a taste-masking membrane comprising a water-insoluble polymer or a water-insoluble polymer in combination with a gastrosoluble pore-forming agent, over the IR microparticles;
preparing organic acid cores;
coating the organic acid cores with an SR or TPR coating comprising a water-insoluble polymer alone or a water-insoluble polymer in combination with a water-soluble polymer or an enteric polymer at a ratio of from about 95:5 to about 50:50 for a coating weight of up to about 30%, to provide a sustained-release profile;
layering the selective serotonin 5-HT₃ blocking agent or pharmaceutically acceptable salt thereof from a polymer binder solution onto the SR- or TPR-coated organic acid cores and optionally applying a protective seal-coat with a water-soluble polymer;
optionally applying a barrier (SR) coating of a water-insoluble polymer, or a water-insoluble polymer in combination with a water-soluble polymer at a ratio of from about 95:5 to about 50:50 to the selective serotonin 5-HT₃ blocking agent layered beads for a weight gain of from about 1.5% to 20% by dry weight of the coated IR beads;
applying an outer lag-time coating to the selective serotonin 5-HT₃ blocking agent layered beads, wherein the outer lag-time coating comprises a water-insoluble polymer in combination with an enteric polymer at a ratio of from about 9:1 to 1:3 for a weight gain of from about 10% to 60% by weight of the coated bead; and
preparing rapidly dispersing microgranules comprising a super disintegrant, at least one sugar alcohol, a saccharide, or a mixture thereof, each primary particle having an average particle size of not more than 30 μm by high shear granulation and fluid bed drying or tray drying;
blending/compressing appropriate amounts of each of rapidly dispersing microgranules, taste-masked IR microparticles, TPR beads, and other pharmaceutically acceptable excipients, into ODT tablets.

30. The method of claim 29, wherein said compressing is with an external lubrication system for externally lubricating dies and punches prior to each compression

31. The method of claim 29, wherein each of said SR- or TPR-coating, drug-layering and outer lag-time coating is applied from a solution in a pharmaceutically acceptable solvent system or from an aqueous dispersion.

32. The method of claim 29 wherein the ODT comprises therapeutically effective amounts of a weakly basic, selective serotonin 5-HT₃ blocking agent as taste-masked IR drug beads and one or more TPR bead populations, said TPR bead population exhibiting differing release characteristics following a pre-determined lag-time.

33. A method of treating or preventing postoperative nausea and vomiting or postdischarge nausea and vomiting for up to 24 hours post-dosing, comprising orally administering to a surgical patient in need thereof, at least one ODT of claim 1, prior to and/or after surgery and optionally once-daily for up to 4 additional days.

34. A method of treating or preventing nausea and vomiting for up to 24 hours post-dosing, comprising orally administering to a cancer patient in need thereof, at least one ODT tablet of claim 1, prior to undergoing moderately emetogenic cancer chemotherapy and optionally once-daily for up to 2 additional days.

35. A method of treating or preventing nausea and vomiting associated with radiotherapy in patients receiving either total body irradiation, single high-dose fraction to the abdomen, or daily fractions to the abdomen for up to 24 hours post-dosing, comprising orally administering to a cancer patient in need thereof, at least one ODT tablet of claim 1 prior to radiotherapy and optionally once-daily for up to 2 additional days.

36. The method of claim 29, wherein the selective serotonin 5-HT₃ blocking agent is ondansetron or pharmaceutically acceptable salts and/or solvates thereof, in an amount equivalent to 16 to 24 mg of ondansetron as base.
